(19) United States
(12) Patent Application Publication
(10) Pub. No.: US 2017/0158745 A1
(43) Pub. Date: Jun. 8, 2017

(54) IDR PEPTIDE COMPOSITIONS AND USE THEREOF FOR TREATMENT OF TH2-DYSREGULATED INFLAMMATORY CONDITIONS

(71) Applicant: University of Manitoba, Winnipeg (CA)

(72) Inventor: Neeloffer MOOKHERJEE, Winnipeg (CA)

(21) Appl. No.: 15/100,213

(22) PCT Filed: Nov. 27, 2014
(86) PCT No.: PCT/CA2014/051136

§ 371 Date: May 27, 2016

Related U.S. Application Data
(60) Provisional application No. 61/910,093, filed on Nov. 28, 2013.

(51) Int. Cl. C07K 14/47; C07K 7/08

(52) U.S. Cl. CPC C07K 14/4723; C07K 7/08; A61K 38/00

(57) ABSTRACT

A composition for treating or preventing a Th2-dysregulated inflammatory condition wherein the composition comprises a therapeutically effective amount of one or more IDR peptides. Use of IDR peptides or a composition comprising IDR peptides for treating or preventing a Th2-dysregulated inflammatory condition. The Th2-dysregulated inflammatory condition includes allergy or atopy, for example allergic asthma.

### FIELD OF THE INVENTION

The present invention relates to the field of Th2-dysregulated inflammatory diseases and, in particular, to compositions comprising an innate defence regulator (IDR) peptide to treat Th2-dysregulated inflammatory conditions.

### BACKGROUND OF THE INVENTION

A Th2-dysregulated immune response results from an imbalance between two subpopulations of T-lymphocytes, type 1 and type 2 helper T-cells (Th1 and Th2 cells). A Th2-biased response to environmental allergens can result in a number of common human diseases most notably atopy, allergy, and autoimmunity. Such disorders are generally characterized by an increased ability of lymphocytes to produce IgE antibodies in response to ubiquitous antigens. Activation of the immune system by these antigens leads to allergic inflammation and may occur after ingestion, penetration through the skin, or after inhalation. When this immune activation occurs and pulmonary inflammation ensues this disorder is broadly characterized as asthma.

Allergic asthma is a Th2-polarized chronic inflammatory disease that is characterized by airflow obstruction, airway hyper-responsiveness (AHR) and airway inflammation. Asthma is typically orchestrated by activation of innate immune cells by allergens followed by an exacerbated Th2-biased inflammation and synthesis of allergen-specific IgE antibody, which initiates the release of histamines and leukotrienes from mast cells. The disease is primarily driven by exposure to allergens such as pollen or HDM (Dermatophagoides sp).

Some of the key cytokines elevated during the pathogenesis of asthma are IL-4, IL-5 and IL-13 and these cytokines are thought to be essential in the development in AHR, mucus production, immunoglobulin class switching to IgE, and survival of predominant airway inflammatory infiltrate, eosinophils. CCL11, CCL24, CCL22, CCL17 are chemokines that have been shown to promote eosinophils and Th2 cells to the lung and contribute to the Th2 inflammatory response.

Th2-dysregulated inflammatory diseases such as allergic asthma impose a devastating burden worldwide affecting nearly 300 million and ˜3 million Canadians (www.publichealth.gc.ca). Accordingly, there remains a need for new and effective therapeutic strategies for managing Th2-dysregulated inflammatory diseases, such as allergy and asthma.

Host defence peptides (HDPs) are natural peptides which can control both infection and pathogen influenced inflammation, as well as maintain immune homeostasis. Synthetic versions of these HDPs, known as innate defense regulator (IDR) peptides, have been shown to have reduced cytotoxicity compared to HDPs and are designed to exhibit optimized immune-modulatory activity. Previous studies have shown that IDR peptides suppress pathogen-induced inflammation and confer protection against a variety of infections (Bowdish, D. M. et al. Impact of LL-37 on anti-infective immunity. Journal of leukocyte biology 77, 451-459 (2005), Mookherjee, N., Rehaume, L. M. & Hancock, R. E. Cathelicidins and functional analogues as antisepsis molecules. Expert opinion on therapeutic targets 11, 993-1004 (2007), Nijnik, A. et al. Synthetic cationic peptide IDR-1002 provides protection against bacterial infections through chemokine induction and enhanced leukocyte recruitment. Journal of immunology 184, 2539-2550 (2010)). This dual ability of IDR peptides to control infections and suppress inflammation makes them attractive therapeutic agents; as well, the safety of IDR peptides for human use has been established (Hancock, R. E., Nijnik, A. & Philpott, D. J. Modulating immunity as a therapy for bacterial infections. Nature reviews. Microbiology 10, 243-254 (2012), Cherkasov, A. et al. Use of artificial intelligence in the design of small peptide antibiotics effective against a broad spectrum of highly antibiotic-resistant superbugs. ACS chemical biology 4, 65-74 (2009)).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

According to embodiments of the present disclosure, there is described herein IDR peptide compositions and uses thereof for treatment of Th2-dysregulated inflammatory conditions. In accordance with one aspect of the disclosure, there is described a pharmaceutical composition for treating or preventing a Th2-dysregulated inflammatory condition in a subject, the composition comprising a therapeutically effective amount of one or more IDR peptides, and a pharmaceutically acceptable carrier.

In accordance with another aspect of the disclosure, there is described a method of treating or preventing a Th2-dysregulated inflammatory condition in a subject comprising administering to the subject an effective amount of one or more IDR peptides or the composition described herein.

In accordance with a further aspect of the disclosure, there is described a use of one or more IDR peptides or the composition described herein, to treat or prevent a Th2-dysregulated inflammatory condition in a subject in need thereof.

In accordance with another aspect of the disclosure, there is described a use of one or more IDR peptides or the composition described herein in the manufacture of a medicament for treating or preventing a Th2-dysregulated inflammatory condition in a subject.

In accordance with a further aspect of the disclosure, there is described a kit for the administration of one or more IDR peptides or the composition described herein, for treating or preventing a Th2-dysregulated inflammatory condition in a subject, comprising: (i) the one or more IDR peptides or the composition according to any one of claims 1 to 5, either lyophilized or in solution; (ii) contained in a container, such as a syringe, pipette, eye dropper, vial, nasal spray, or inhaler; and (iii) instructions for use.

### BRIEF DESCRIPTION OF THE DRAWINGS

These and other features of the invention will become more apparent in the following detailed description in which reference is made to the appended drawings.

FIG. 1 is a schematic representation of the protocol for preparing an HDM-challenged mouse model of asthma, according to embodiments of the present disclosure;

FIG. 2 is a graphical presentation of the effect of IDR peptide on the lung mechanics in an HDM-challenged mouse model of acute asthma, according to embodiments of the present disclosure. Specifically, FIG. 2A illustrates the effect of an intranasally (i.n.) administered IDR peptide on airway resistance in an HDM-challenged mouse model of acute asthma; FIG. 2B illustrates the effect of a subcutaneously (s.c.) administered IDR peptide on airway resistance in an HDM-challenged mouse model of acute asthma; FIG. 2C illustrates the effect of an intranasally (i.n.) administered IDR peptide on tissue resistance in an HDM-challenged mouse model of acute asthma; and FIG. 2D illustrates the effect of a subcutaneously (s.c.) administered IDR peptide on tissue resistance in an HDM-challenged mouse model of acute asthma;

FIG. 3 is a graphical presentation of the effect of IDR peptide on the lung mechanics in an HDM-challenged mouse model of chronic asthma, according to embodiments of the present disclosure. Specifically, FIG. 3A illustrates the effect of a subcutaneously (s.c.) administered IDR peptide on airway resistance in an HDM-challenged mouse model of chronic asthma; FIG. 3B illustrates the effect of a subcutaneously (s.c.) administered IDR peptide on tissue resistance in an HDM-challenged mouse model of chronic asthma; FIG. 3C illustrates the effect of a subcutaneously (s.c.) administered IDR peptide on tissue elastance in an HDM-challenged mouse model of chronic asthma;

FIG. 4 is a graphical presentation of the effect of IDR peptide on Th2 cytokine IL-33 production in the bronchoalveolar lavage fluid (BALF) and lung tissue in an HDM-challenged murine model of acute allergic asthma wherein individual circles represent a single mouse, according to embodiments of the present disclosure;

FIG. 5 is a graphical presentation of the effect of IDR peptide on Th2 cytokine IL-13 production in the lung tissue in an HDM-challenged murine model of acute allergic asthma wherein individual circles represent a single mouse, according to embodiments of the present disclosure;

FIG. 6 is a graphical presentation of the effect of IDR peptide on HDM-specific IgE in an HDM-challenged murine model of allergic asthma wherein individual circles represent a single mouse, according to embodiments of the present disclosure; and

FIG. 7 is a graphical presentation of the effect of IDR peptide on the infiltration of neutrophils and the production of inflammatory cytokine IL-33 in the lungs of an HDM-challenged mouse model of acute asthma, according to embodiments of the present disclosure. Specifically, FIG. 7A illustrates the effect of a subcutaneously (s.c.) administered IDR peptide on neutrophil numbers in the bronchoalveolar lavage fluid (BALF); and FIG. 7B illustrates the effect of a subcutaneously (s.c.) administered IDR peptide on cytokine IL-33 production in lung tissue.

### DETAILED DESCRIPTION OF THE INVENTION

Due to their optimized immune-modulatory properties and reduced cytotoxicities, IDR peptides have been considered for a variety of therapeutic applications. Specifically, IDR peptides have been shown to be effective in the therapy and prophylaxis of infections (Nijnik, A. et al. Synthetic cationic peptide IDR-1002 provides protection against bacterial infections through chemokine induction and enhanced leukocyte recruitment. Journal of immunology 184, 2539-2550 (2010)), Th1-dysregulated inflammatory diseases such as rheumatoid arthritis (Turner-Brannen, E. et al. Modulation of interleukin-1beta-induced inflammatory responses by a synthetic cationic innate defence regulator peptide, IDR-1002, in synovial fibroblasts. Arthritis research & therapy 13, R129 (2011)), and in attenuating hyperinflammatory cytokine production in cystic fibrosis airway cells (Mayer, M. L. et al. Rescue of Dysfunctional Autophagy Attenuates Hyperinflammatory Responses from Cystic Fibrosis Cells. Journal of immunology (2012)). The exemplary embodiments described herein, relate to the determination that IDR peptides are capable of suppressing Th-2 polarized inflammatory cytokines, and allergen-specific antibodies. This capability of IDR peptides have further been shown to result in an improvement in the symptoms associated with Th2-dysregulated inflammatory conditions. IDR peptides, according to the present disclosure, may therefore be useful in the treatment or prevention of various Th2-dysregulated inflammatory conditions, in particular Th-2 polarized inflammatory conditions, for which suppression of Th-2 polarized inflammatory cytokines, and allergen-specific antibodies in the subject is required.

According to embodiments of the present disclosure, IDR peptides are capable of suppressing Th-2 polarized inflammatory cytokines, and allergen-specific antibodies when administered to a subject. In preferred embodiments, IDR peptides are capable of suppressing cytokines IL-33 and/or IL-13, and/or suppressing allergen-specific antibody IgE when administered to a subject. IDR peptides according to embodiments described herein are capable of being used in the treatment or prevention of various Th2-dysregulated inflammatory conditions, in particular Th-2 polarized inflammatory conditions, for which suppression of Th-2 polarized inflammatory cytokines, and allergen-specific antibodies in the subject is required.

IDR peptides according to embodiments of the present disclosure are capable of controlling or treating Th2-dysregulated inflammatory conditions when administered to a subject. In preferred embodiments, IDR peptides are capable of controlling or treating allergy or atopy, for example allergic asthma, when administered to a subject. In further embodiments, IDR peptides are capable of improving lung function when administered to a subject with acute or chronic allergic asthma. In certain embodiments, IDR peptides may be useful in treating or preventing severe steroid-resistant asthma.

### DEFINITIONS

Unless defined otherwise, all technical and scientific terms used herein have the same meaning as commonly understood by one of ordinary skill in the art to which this invention belongs.

As used herein, the term “about” refers to an approximately +/−10% variation from a given value. It is to be understood that such a variation is always included in any given value provided herein, whether or not it is specifically referred to.

“Naturally-occurring,” as used herein, as applied to an object, refers to the fact that the object can be found in nature. For example, a polypeptide or polynucleotide sequence that is present in an organism that can be isolated from a source in nature and which has not been intentionally modified by man in the laboratory is naturally-occurring.

The term “amino acid” means one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides”. A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “treat”, and grammatical variations thereof, means any administration of a compound or composition, of the present invention. Treatment may have a prophylactic effect, a therapeutic effect, or a combination thereof. Treatment can be accomplished using various methods depending on the subject to be treated including, but not limited to, parenteral administration, such as intraperitoneal injection (i.p.), intravenous injection (i.v.) or intramuscular injection (i.m.); oral administration; intranasal administration (i.n.); intradermal administration; subcutaneous administration (s.c.); transdermal administration and immersion.

The term “subject” or “patient” as used herein refers to an animal in need of treatment, and specifically includes humans. The term “animal,” as used herein, refers to both human and non-human animals, including, but not limited to, mammals, birds and fish, and encompasses domestic, farm, zoo, laboratory and wild animals, such as, for example, cows, pigs, horses, goats, sheep or other hoofed animals, dogs, cats, chickens, ducks, non-human primates, guinea pigs, rabbits, ferrets, rats, hamsters and mice. Accordingly, the term “subject” or “patient” as used herein means any patient or subject to which the compounds or compositions of the disclosure can be administered. In an exemplary aspect of the present disclosure, to identify subject patients for treatment with a compound or pharmaceutical composition of the present disclosure, accepted screening methods are employed to determine the status of an existing disease or condition in a subject or risk factors associated with a targeted or suspected disease or condition. These screening methods include, for example, examinations to determine whether a subject is suffering from a Th2-dysregulated inflammation disease or disorder. These and other routine methods allow the clinician to select subjects in need of therapy.

The term “substantially identical,” as used herein in relation to a nucleic acid or amino acid sequence indicates that, when optimally aligned, for example using the methods described below, the nucleic acid or amino acid sequence shares at least 70%, at least 75%, at least 80%, at least 85%, at least 90%, at least 95%, at least 96%, at least 97%, at least 98% or at least 99% sequence identity with a defined second nucleic acid or amino acid sequence (or “reference sequence”). “Substantial identity” may be used to refer to various types and lengths of sequence, such as full-length sequence, functional domains, coding and/or regulatory sequences, promoters, and genomic sequences. Percent identity between two amino acid or nucleic acid sequences can be determined in various ways that are within the skill of a worker in the art, for example, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool (Altschul, S. F., W. Gish, et al. (1990) J Mol Biol 215: 403-10), and variations thereof including BLAST-2, BLAST-P, BLAST-N, BLAST-X, WU-BLAST-2, ALIGN, ALIGN-2, CLUSTAL, and Megalign (DNASTAR) software. In addition, those skilled in the art can determine appropriate parameters for measuring alignment, including algorithms needed to achieve maximal alignment over the length of the sequences being compared. In general, for amino acid sequences, the length of comparison sequences will be at least 10 amino acids. One skilled in the art will understand that the actual length will depend on the overall length of the sequences being compared and may be at least 20, at least 30, at least 40, at least 50, at least 60, at least 70, at least 80, at least 90, at least 100, at least 110, at least 120, at least 130, at least 140, at least 150, or at least 200 amino acids, or it may be the full-length of the amino acid sequence. For nucleic acids, the length of comparison sequences will generally be at least 25 nucleotides, but may be at least 50, at least 100, at least 125, at least 150, at least 200, at least 250, at least 300, at least 350, at least 400, at least 450, at least 500, at least 550, or at least 600 nucleotides, or it may be the full-length of the nucleic acid sequence.

### Innate Defense Regulator (IDR) Peptides

IDR peptides are synthetic variants of the naturally-occurring host defense peptides (HDPs). Naturally-occurring HDPs are cationic amphipathic molecules with immunomodulatory and microbicidal properties. Many HDPs have been characterized of which the defensins and cathelicidins have been of greatest focus. There are more than 1500 HDPs (http://aps.unmc.edu/AP/main.php) currently identified which have provided templates for designing short synthetic peptides, using internal fragments or amino acid substitutions, for designing IDR peptides; and using known methods, IDR peptides have been developed and optimized to exhibit improved immune-modulatory properties and reduced cytotoxicities compared to the parent HDP.

According to embodiments of the present disclosure, the therapeutic use of IDR peptides for treating or preventing a Th2-dysregulated inflammatory condition is described. In preferred embodiments, the Th2-dysregulated inflammatory condition is atopy or allergy, for example allergic asthma. As well, the use of the IDR peptides for the preparation of medicaments and/or pharmaceutical compositions is within the scope of the present disclosure.

IDR peptides, according to embodiments of the present disclosure, include an IDR peptide that is a synthetic variant of an HDP that includes for example defensin, cathelicidin, magainin, melittin, cecropin, bactenecin, indolicidin, polyphemusin, and tachyplesin. In other embodiments, the IDR peptide is a synthetic variant of the HDP bactenecin. In preferred embodiments, the IDR peptide is the HDP bactenecin derivative IDR-1002 which is known in the art and is provided herein as SEQ ID NO:1 (VQRWLIVWRIRK-NH2).

IDR peptides, according to embodiments of the present disclosure, include analogs and derivatives of the IDR peptides described herein provided that the peptide retains immune-modulatory activity. For example, the IDR peptides may be mutagenized by substitution, insertion or deletion of one or more amino acid residues so that the residue at that site does not correspond to the parental (reference) sequence. One skilled in the art will appreciate, however, that peptides comprising such mutations will still retain immune-modulatory activity.

In certain embodiments, IDR peptides include fragments of the IDR peptides described herein provided that the peptide retains immune-modulatory activity. For example, a fragment may comprise a deletion of one or more amino acids from the N-terminus, the C-terminus, or the interior of the protein, or a combination thereof so long as the peptide retains immune-modulatory activity.

In certain embodiments of the present disclosure, when an IDR peptide comprises a variant sequence, the variant sequence is at least about 70% identical to the parental (reference) sequence, for example, at least about 75% identical to the reference sequence. In some embodiments, the variant sequence is at least about 80%, at least about 85%, at least about 90%, at least about 95%, at least about 97% identical, and at least about 98% identical to the reference sequence. In one embodiment, the reference amino acid sequence is SEQ ID NO:1.

### Evaluation of Efficacy

The efficacy of the IDR peptides for use in treating or preventing a Th2-dysregulated inflammatory condition can be assessed by various standard in vitro and in vivo techniques known in the art, including those described in the Examples presented herein.

### IDR Peptide Compositions

The present disclosure describes compositions suitable for use in treating or preventing a Th2-dysregulated inflammatory condition. Compositions according to the present disclosure comprise one or a combination of two or more IDR peptides described herein, together with one or more pharmaceutically acceptable carriers, diluents and/or excipients. If desired, other active ingredients, adjuvants and/or immunopotentiators may be included in the compositions.

The compositions can be formulated for administration by a variety of routes. For example, the compositions can be formulated for oral, topical, rectal, nasal or parenteral administration or for administration by inhalation or spray. The term parenteral as used herein includes subcutaneous injections, intravenous, intramuscular, intrathecal, intrasternal injection or infusion techniques. Intranasal administration to the subject includes administering the pharmaceutical composition to the mucous membranes of the nasal passage or nasal cavity of the subject. In one embodiment of the present invention, the compositions are formulated for topical, rectal or parenteral administration or for administration by inhalation or spray, for example by an intranasal route. In another embodiment, the compositions are formulated for parenteral administration.

The compositions preferably comprise an effective amount of the one or more IDR peptides. The term “effective amount” as used herein refers to an amount of the IDR peptides required to induce suppression of Th-2 polarized inflammatory cytokines, and/or allergen-specific antibodies. The effective amount of the IDR peptides for a given indication can be estimated initially, for example, either in cell culture assays or in animal models, usually in rodents, rabbits, dogs, pigs or primates. The animal model may also be used to determine the appropriate concentration range and route of administration. Such information can then be used to determine useful doses and routes for administration in the animal to be treated, including humans. One or more doses may be used in treating the animal, and these may be administered on the same day or over the course of several days or weeks, for example.

Compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavoring agents, coloring agents and preserving agents in order to provide pharmaceutically elegant and palatable preparations. Tablets contain the multimers in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Compositions for oral use can also be presented as hard gelatine capsules wherein the IDR peptides are mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Compositions for nasal administration can include, for example, nasal spray, nasal drops, suspensions, solutions, gels, ointments, creams, and powders. The compositions can be formulated for administration through a suitable commercially available nasal spray device, such as Accuspray™ (Becton Dickinson). Other methods of nasal administration are known in the art.

Compositions formulated as aqueous suspensions contain the IDR peptides in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Compositions can be formulated as oily suspensions by suspending the IDR peptides in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may optionally be added to provide palatable oral preparations. These compositions can be preserved by the addition of an anti-oxidant such as ascorbic acid.

The compositions can be formulated as a dispersible powder or granules, which can subsequently be used to prepare an aqueous suspension by the addition of water. Such dispersible powders or granules provide the multimers in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

Compositions can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavouring agents.

Compositions can be formulated as a syrup or elixir by combining the IDR peptides with one or more sweetening agents, for example glycerol, propylene glycol, sorbitol or sucrose. Such formulations can also optionally contain one or more demulcents, preservatives, flavouring agents and/or colouring agents.

The compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the composition of the present invention may contain preservatives such as antimicrobial agents, anti-oxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum or skimmed milk) together with a suitable buffer (e.g. phosphate buffer). The pH and exact concentration of the various components of the composition may be adjusted according to well-known parameters.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

### Kits

The present invention additionally provides for kits comprising one or more IDR peptides for use as a medicament for treating a Th2-dysregulated inflammatory condition. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the medicament.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, nasal spray device, syringe, pipette, forceps, measured spoon, eye dropper or similar medically approved delivery vehicle.

To gain a better understanding of the invention described herein, the following examples are set forth. It will be understood that these examples are intended to describe illustrative embodiments of the invention and are not intended to limit the scope of the invention in any way.

### EXAMPLES

In the Examples section, the efficacy of an IDR peptide on a Th2-dysregulated inflammatory condition is described. An exemplary IDR peptide (IDR-1002) was examined in a murine model of allergic asthma and the results described herein.

The peptide IDR 1002 is a 12-amino acid α-helical peptide (VQRWLIVWRIRK-NH2, SEQ ID NO: 1) and a synthetic derivative of a bovine HDP, bactenecin. IDR 1002 has been shown in murine models of infections to modulate host immune responses, mainly by inducing chemokines and recruiting immune cells to the site of infection to protect against various pathogens (Nijnik, A. et al. Synthetic cationic peptide IDR-1002 provides protection against bacterial infections through chemokine induction and enhanced leukocyte recruitment. Journal of immunology 184, 2539-2550 (2010), Rivas-Santiago, B. et al. Ability of Innate Defence Regulator Peptides IDR-1002, IDR-HH2 and IDR-1018 to Protect against Mycobacterium tuberculosis Infections in Animal Models. PLoS One 8, e59119 (2013)). IDR 1002 was obtained from CPC Scientific (CA, USA) and was re-suspended in saline for use.

### Example 1: Effect of IDR Peptide on Lung Function in a HDM-Challenged Murine Model of Allergic Asthma

### Methods

### House Dust Mite (HDM)-Challenged Murine Model of Allergic Asthma

A House Dust Mite (HDM)-challenged murine model of allergic asthma was used. This murine model results in Th2-polarized bronchial inflammation, airway remodeling and epithelial damage similar to that seen in human asthma. Based on the duration of HDM exposure, the model can either represent the acute or chronic stage of allergic airway inflammation. The acute phenotype of airway inflammation occurs during the first two weeks of HDM exposure, and generally involves significant levels of inflammation. This includes recruitment of immune cells and production of pro-inflammatory cytokines in the lung. Chronic phenotype is achieved through continuous and receptive exposure of the allergen (5 weeks), which causes permanent changes in structural cells. This results in nearly irreversible narrowing of airways, through smooth muscle and epithelial cell hyperplasia, lung fibrosis, collagen accumulation etc. Collectively these processes are referred to as airway remodeling.

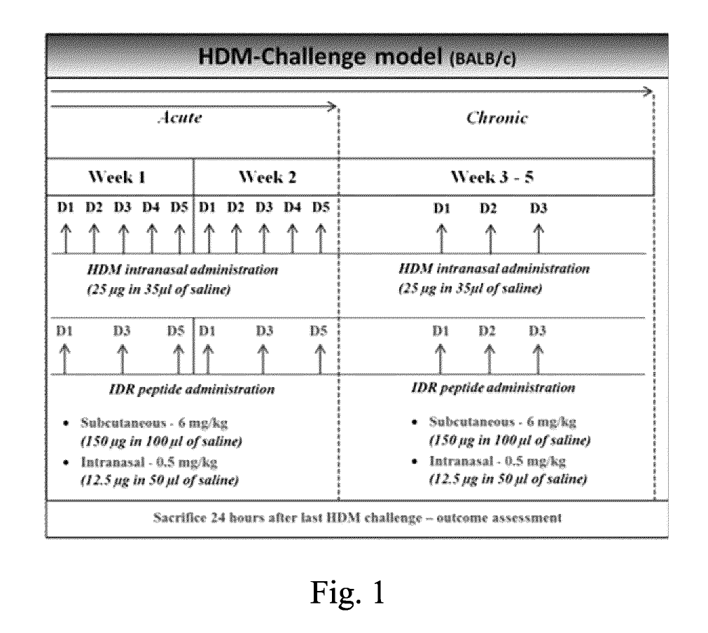

Referring to FIG. 1, which shows a schematic representation of the protocol for preparing an HDM-challenged mouse model of asthma, 8-10 week old female Balb/C mice were challenged with 5 consecutive intranasal (i.n) administrations of whole HDM extract during week 1 and 2 to represent the acute phase. Specifically, on Day 1 (D1) Female Balb/c mice (8-10 weeks of age) were challenged with 35 μL of whole house dust mite (HDM) extract (0.7 mg/mL in saline) with five daily i.n. administrations in week 1 and 2 (acute protocol).

To represent the chronic phase of the experiment, mice were challenged for an additional 3 weeks, where HDM was administered i.n on days 1, 3, and 5 only. Specifically, for the chronic protocol the animals were maintained for an additional three weeks with three daily mid-week i.n. HDM challenges (total of 5 weeks).

IDR 1002 was administered either by subcutaneous (s.c) injections (6 mg/kg) or by i.n. administration (0.5 mg/kg), on days 1, 3 and 5 of every week. For all experiments, age matched naïve mice were used as controls. Animals were assessed at two time points: (i) ˜24 hr after the acute phase, end of week 2, and (ii) ˜24 hr after the chronic phase, end of week 5.

### Measurement of Lung Function

Lung function was measured using a flexiVent™ small animal ventilator. Briefly, mice were anesthetized using sodium pentobarbital, followed by tracheal surgery, where a catheter is inserted to the trachea. The flexiVent™ small animal ventilator measures respiratory mechanics using forced oscillation, during which it measures volume displaced by the piston and the pressure in the cylinder. From this raw data, using known complex algorithms, airway resistance, tissue resistance and tissue elastance was calculated (http://www.scireq.com/products/flexivent/). Mice were exposed to increasing dose methacholine, a bronchoconstrictor, and changes in the above stated parameters were monitored.

### Results

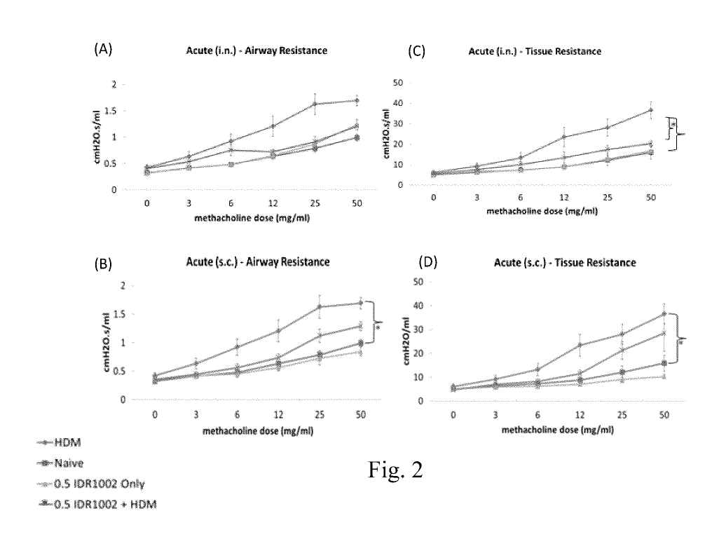

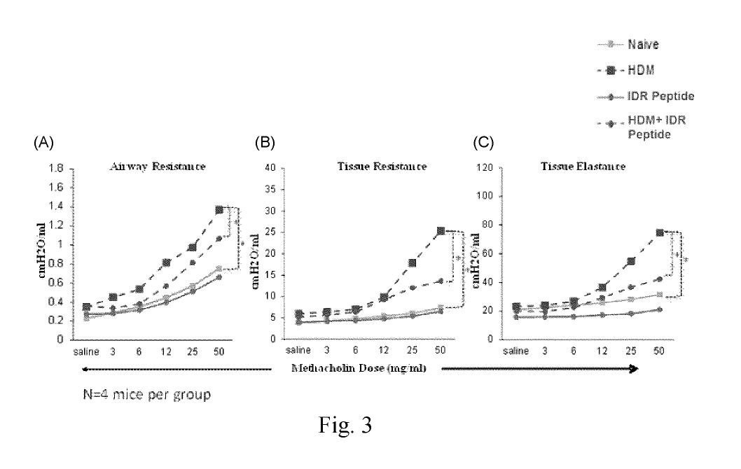

Airway resistance, tissue resistance and elastance to inhaled methacholine are presented in FIGS. 2 and 3. As shown, HDM challenge significantly increased airway resistance compared to naïve mice. I.n. and s.c. administration of IDR 1002 significantly reduced airway resistance in HDM-challenged mice in the acute (FIGS. 2A and B, respectively) and chronic model (FIG. 3A). Tissue resistance was significantly reduced by i.n. (FIG. 2C), but not during s.c. (FIG. 2D), administration of IDR 1002 in HDM-challenged mice. (*p<0.05).

Based on the above observations, HDM-challenged mice were shown to have had increased airway and tissue resistance to inhaled methacholine compared to naïve mice. Administration of IDR 1002 i.n. and s.c. significantly reduced these responses in acute HDM-challenged mice (FIG. 2). The i.n administration of the peptide had more robust effect, and improved both airway resistance and tissue resistance in HDM-challenged mice. This suggests that route of delivery of the peptide i.e. systemic (s.c.) v/s local (i.n.) may have different effects on the endpoint outcomes.

In a chronic HDM-challenged murine model, subcutaneously (s.c.) administered IDR 1002 was shown to significantly improve lung function (airway resistance, tissue resistance and tissue elastance) in HDM-challenged mice (FIG. 3).

### Example 2: Effect of IDR Peptide on Th2 Cytokines in HDM-Challenged Murine Lung Tissue

### Methods

Cytokines IL-33 and IL-13 levels in bronchoalveolar lavage fluid, serum and the lung protein extracts (50 μg total protein per extract) were monitored by ELISA kits purchased from R&D systems.

8-10 wks female Balb/c mice (n=5 per group) were challenged by intranasal administration of 35 μl of whole HDM extract (0.7 mg/ml) in saline, for two weeks to represent the murine model of acute allergic asthma. IDR-1002 was administered either s.c. at a dose of 6 mg/Kg or i.n. at a dose of 0.5 mg/Kg per mouse, three administrations per week. Lung tissue was collected 24 hours after the last HDM challenge. IL-33 levels were then determined.

To determine levels of IL-13, 8-10 wks female Balb/c mice (n=5 per group) were challenged by intranasal administration of 35 μl of whole HDM extract (0.7 mg/ml) in saline, for two weeks (acute) and 5 weeks (chronic). IDR-1002 was administered s.c. at a dose of 6 mg/Kg per mouse, three administrations per week. Lung tissue was collected 24 hours after last HDM challenge.

### Results

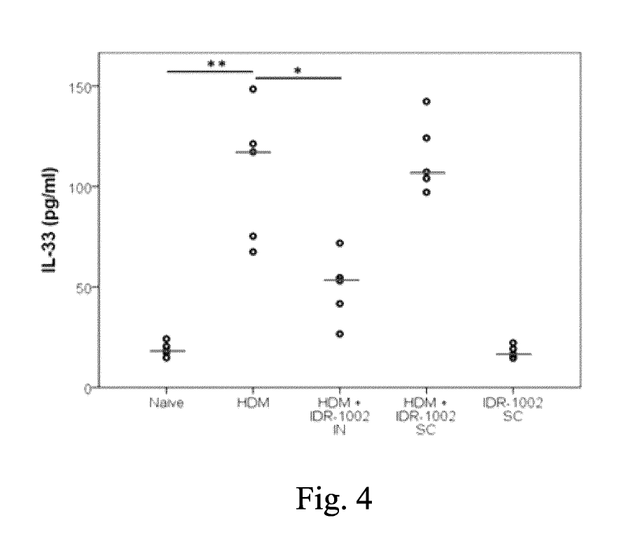

The effect of IDR 1002 on cytokine levels in the BALF and lung tissue of HDM-challenged mice was examined. As shown in FIG. 4, epithelial derived Th2-cytokine IL-33 was suppressed in lung tissue with i.n., but not s.c. administration. IL-33, mainly produced by epithelial cells, is thought to be a key early cytokine responsible for skewing the immune response to a Th2 response as well as induction of IL-13, and is essential for the development of an allergic response.

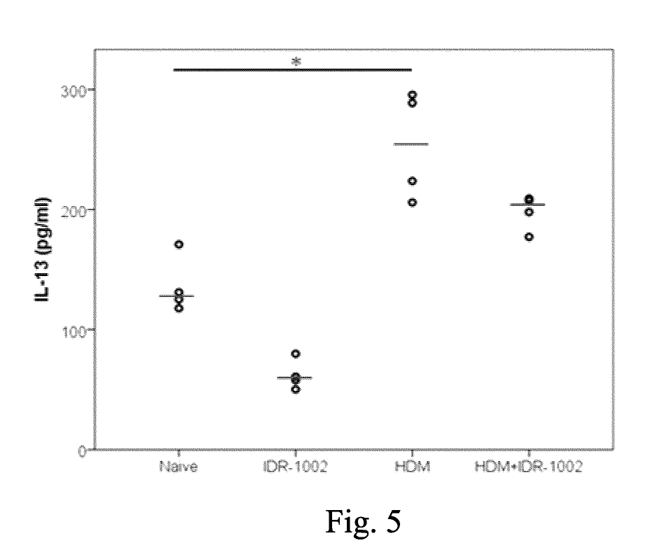

Consistent with this, s.c. administration of IDR 1002 was observed to notably suppress levels of IL-13 in acute HDM-challenged lung tissue. Referring to FIG. 5, 2 week acute HDM-challenged mice were observed to have significantly higher IL-13 in lung tissue compared to naïve (p<0.05). S.c administration of IDR 1002 notably reduced IL-13 levels in HDM-challenged mice.

Furthermore, the peptide IDR 1002 by itself did not induce other pro-inflammatory cytokines such as TNFα, IL-1β, IL-4 or IL-5, either in the BALF, in serum or in the lung tissue extract.

These results, therefore, show that IDR 1002 can suppress Th2 cytokines such as IL-33 and IL-13, both critical in the pathogenesis of allergic asthma. As well, it has been shown that i.n. administration of IDR 1002 suppresses IL-33 in HDM-challenged mice, which is critical in severe chronic asthma.

Moreover, as IL-33 has been identified as a Th2 cytokine (IL-1 family) known to play a significant role in severe steroid resistant asthma, the results further indicate that IDR peptides may be beneficial in the treatment of severe steroid-resistant asthma.

### Example 3: Effect of IDR Peptide on HDM-Specific IgE Antibodies in HDM-Challenged Murine Lung Tissue

Allergic asthma is primarily mediated by production of allergen specific IgE antibodies. IgE antibodies bind to the surface of mast cells and basophils and subsequent exposure to the allergen, results in degranulation of these cells. The mediators released primarily from mast cells, causes many physiological symptoms such as mucus production, airway constriction, vascular dilation etc.

HDM-specific IgE levels were measured in serum by ELISA to determine the effect of IDR peptide on the production of HDM-specific IgE antibodies.

### Methods

Levels of HDM-specific IgE was evaluated by ELISA (purchased from R&D systems) in the serum using Southern biotech antibodies.

HDM-specific IgE production in an HDM-challenged murine model of allergic asthma was measured using 8-10 wks female Balb/c mice (n=5 per group) which were challenged by intranasal administration of 35 μl of whole HDM extract (0.7 mg/ml) in saline, either for two weeks (acute) or 5 weeks (chronic). IDR-1002 was administered s.c. at a dose of 6 mg/Kg, three administrations per week.

### Results

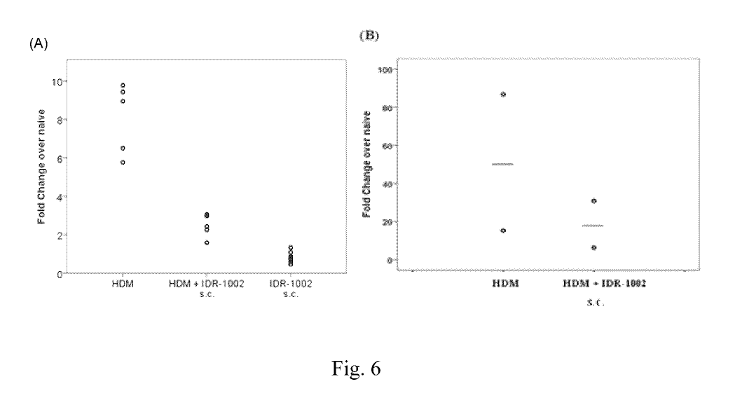

Referring to FIG. 6, HDM specific IgE levels were monitored in the serum and a significantly higher HDM-specific IgE in serum was observed for 2 week acute HDM-challenged mice compared to naïve (p<0.005) (FIG. 6A). S.c. administration of IDR 1002 significantly reduced HDM-specific IgE in HDM-challenged mice (p<0.05).

As shown in FIG. 6B, a notable reduction of HDM-specific IgE with s.c. administration of IDR 1002 in 5 week chronic HDM-challenged mice serum was observed.

Based on the above observations, it has been demonstrated that s.c. administration of an IDR peptide (IDR 1002) can suppress HDM-specific IgE antibodies in the serum of 2 week acute (FIG. 6A) and in 5 week chronic HDM-challenged mice (FIG. 6B).

### Example 4: Effect of IDR Peptide on Steroid-Resistant Asthma

10% of asthma patients are steroid-refractory and more than 50% of health care costs is related to this subgroup of severe asthma patients (Caramori, G., Groneberg, D., Ito, K., Casolari, P., Adcock, I. M. & Papi, A. New drugs targeting Th2 lymphocytes in asthma. J Occup Med Toxicol 3 Suppl 1, S6 (2008)).

The cytokine IL-33 is a steroid-resistant mediator promoting Th2-immune deviation and airway remodeling in asthma, and therefore is a critical therapeutic target (Farahani, R., Sherkat, R., Hakemi, M. G., Eskandari, N. & Yazdani, R. Cytokines (interleukin-9, IL-17, IL-22, IL-25 and IL-33) and asthma. Adv Biomed Res 3, 127 (2014); Saglani, S., Lui, S., Ullmann, N., Campbell, G. A., Sherburn, R. T., Mathie, S. A., Denney, L., Bossley, C. J., Oates, T., Walker, S. A., Bush, A. & Lloyd, C. M. IL-33 promotes airway remodeling in pediatric patients with severe steroid-resistant asthma. J Allergy Clin Immunol 132, 676-685 e613 (2013)).

In order to assess the effect of IDR peptide on steroid-resistant asthma, neutrophil and IL-33 levels were observed.

### Methods

8-10 wks female Balb/c mice were challenged by intranasal administration of 35 μl of whole HDM extract (0.7 mg/ml) in saline, for two weeks (acute). IDR-1002 was administered subcutaneously (6 mg/Kg per mouse), three administrations per week. Neutrophil numbers were quantified in the bronchoalveolar lavage fluid (BALF) using a modified Wright-Giemsa staining (Hema Stat Pack). Lung tissue was collected 24 hours after the last HDM challenge and the lung homogenates were monitored for cytokine IL-33 by ELISA.

### Results

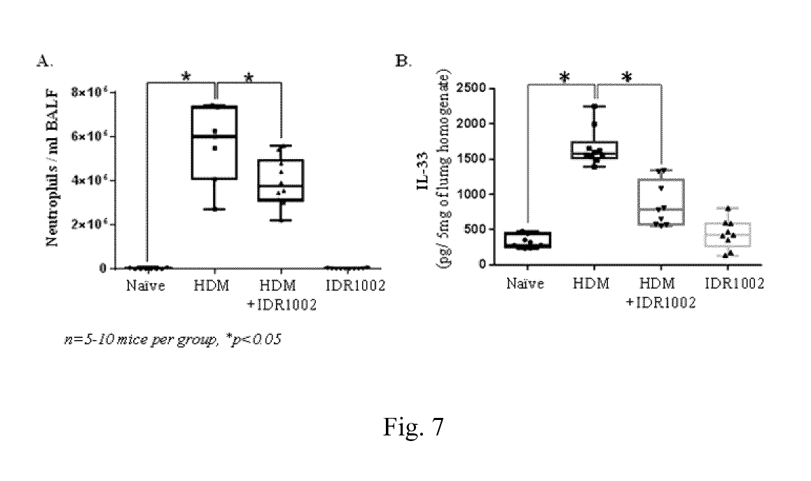

As shown in FIGS. 7A and 7B, respectively, IDR-1002 significantly decreased infiltration of neutrophils and the production of inflammatory cytokine IL-33, in the lungs of the HDM-challenged mice in the acute (2-week) model.

As both neutrophilia and IL-33 are associated with resistance to steroids (Saglani, S., Lui, S., Ullmann, N., Campbell, G. A., Sherburn, R. T., Mathie, S. A., Denney, L., Bossley, C. J., Oates, T., Walker, S. A., Bush, A. & Lloyd, C. M. IL-33 promotes airway remodeling in pediatric patients with severe steroid-resistant asthma. J Allergy Clin Immunol 132, 676-685 e613 (2013), Ano, S., Morishima, Y., Ishii, Y., Yoh, K., Yageta, Y., Ohtsuka, S., Matsuyama, M., Kawaguchi, M., Takahashi, S. & Hizawa, N. Transcription factors GATA-3 and RORgammat are important for determining the phenotype of allergic airway inflammation in a murine model of asthma. J Immunol 190, 1056-1065 (2013)), these results suggest that IDR peptides may be beneficial in the control of severe steroid-refractory asthma.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

The scope of the claims should not be limited by the preferred embodiments set forth in the examples, but should be given the broadest interpretation consistent with the description as a whole.

1. A pharmaceutical composition for treating or preventing a Th2-dysregulated inflammatory condition in a subject, the composition comprising a therapeutically effective amount of one or more IDR peptides, and a pharmaceutically acceptable carrier.

2. The pharmaceutical composition according to claim 1, wherein the one or more IDR peptides comprises a synthetic variant of an HDP selected from the group consisting of defensin, cethelicidin, magainin, melittin, cecropin, bactenecin, indolicidin, polyphemusin, and tachyplesin.

3. The pharmaceutical composition according to claim 1, wherein the one or more IDR peptides comprises a synthetic variant of the HDP bactenecin.

4. The pharmaceutical composition according to claim 1, wherein the one or more IDR peptides shares at least 80% sequence identity with SEQ ID NO:1.

5. The pharmaceutical composition according to claim 1, wherein the one or more IDR peptides comprises SEQ ID NO:1.

6. The pharmaceutical composition according to claim 1, wherein the Th2-dysregulated inflammatory condition is allergy or atopy.

7. The method according to claim 6, wherein the allergy or atopy is allergic asthma.

8. The method according to claim 7, wherein the allergic asthma is a steroid-resistant asthma.

9. A method of treating or preventing a Th2-dysregulated inflammatory condition in a subject comprising administering to the subject an effective amount of the composition according to claim 1.

10. The method according to claim 9, wherein the Th2-dysregulated inflammatory condition is allergy or atopy.

11. The method according to claim 10, wherein the allergy or atopy is allergic asthma.

12. The method according to claim 11, wherein the allergic asthma is a steroid-resistant asthma.

13. The method according to claim 9, wherein administering the composition to the subject induces suppression of Th-2 polarized inflammatory cytokines, and/or allergen-specific antibodies.

14. The method according to claim 9, wherein administering the composition to the subject induces suppression of cytokines IL-33 and/or IL-13, and/or suppressing allergen-specific antibody IgE.

15. Use of the composition according to claim 1 to treat or prevent a Th2-dysregulated inflammatory condition in a subject in need thereof.

16. Use of the composition according to claim 1 in the manufacture of a medicament for treating or preventing a Th2-dysregulated inflammatory condition in a subject.

17. The use according to claim 15, wherein the Th2-dysregulated inflammatory condition is an allergy or an atopy.

18-19. (canceled)

20. The use according to claim 15, wherein the composition induces suppression of Th-2 polarized inflammatory cytokines, and/or allergen-specific antibodies.

21. (canceled)

22. A kit for treating or preventing a Th2-dysregulated inflammatory condition in a subject, comprising:
(i) the composition according to claim 1, either lyophilized or in solution;
(ii) contained in a container selected from a group consisting of a syringe, a pipette, an eye dropper, a vial, a nasal sprayer, and a nasal inhaler; and
(iii) instructions for use.

23-25. (canceled)


SEQUENCE LISTING (1 sequence)

SEQ ID NO: 1 - protein, 12 aa, Artificial Sequence
  IDR-1002 peptide derivative
VQRWLIVWRIRK